(19) United States
(12) Patent Application Publication
(10) Pub. No.: US 2017/0119568 A1
(43) Pub. Date: May 4, 2017

(54) AUXILIARY ELASTIC DEVICE FOR JOINT PROTECTOR

(71) Applicant: SPECIAL PROTECTORS CO., LTD., New Taipei City (TW)

(72) Inventors: Chi-Sheng CHIANG, New Taipei City (TW); Chi-Lan LIAO, New Taipei City (TW); Jung-Shun YU, New Taipei City (TW)

(73) Assignee: SPECIAL PROTECTORS CO., LTD., New Taipei City (TW)

(21) Appl. No.: 15/298,361

(22) Filed: Oct. 20, 2016

(30) Foreign Application Priority Data
Nov. 4, 2015 (TW) 104136389

(51) Int. Cl. A61F 5/01; A41D 13/06

(52) U.S. Cl. CPC A61F 5/0123; A41D 13/065; A61F 2005/0165; A61F 2005/0172

(57) ABSTRACT

An auxiliary elastic device for a joint protector is capable of producing auxiliary elasticity for human joints. The device includes a connecting body connected to a multiple of serially and pivotally coupled pivots, a through slot penetrated through the connecting body, and at least one stripe elastic rib movably installed in the through slot and capable of providing an elastic supporting force and an elastic restoring force when human joints are bent, so as to achieve the effect of reducing the burden of the joints.

### FIELD OF THE INVENTION

The present invention relates to an auxiliary elastic device applied to a joint protector and capable of producing an auxiliary elasticity for human joints during activities.

### BACKGROUND OF THE INVENTION

1. Description of the Related Art

The pace of walking starts from striking the heel on the ground, setting the body in a vertical standing posture, pivoting the ankle to let the tip of the toe fall onto the ground, changing the pace to lift the heel until the tip of the toe leaves the ground while swinging the leg forward to prepare for striking the heel on the ground again for the next motion cycle. During the walking process, both legs execute the same motion cycle alternately to achieve the effect of moving forward. In the meantime, the knees are situated at an upright state or a bending state to bear the human body weight, and thus it is better to wear a joint protector to protect users, reduce pressure, and avoid knee injury while doing intensive exercise or receiving limb rehabilitation.

The joint protector is primarily used for supporting, guiding and limiting the range of the knee joint movement when the legs are moving. For various different exercises or rehabilitations, different types of joint protectors are developed. For example, these joint protectors include one having an elastic soft fabric wound into a cylindrical shape and wrapped around a knee joint as disclosed in R.O.C. Pat. No. TW M408396, a protective pad added to the front of the knee patella as disclosed in R.O.C. Pat. No. TW M447791, or those having a plurality of elastic metal strips that can be bent or straightened with the leg movement and can provide auxiliary elastic force and supporting force to reduce the knee pressure during the process of squatting or getting up as disclosed in R.O.C. Pat. No. TWM313535, and U.S. Pat. Nos. US20120004584, U.S. Pat. No. 3,473,527, U.S. Pat. No. 5,261,871, U.S. Pat. No. 4,366,813.

However, the manufacture of the elastic metal strip is limited by the coefficient of elasticity of metal, and the elastic metal strip are manufactured in the form of a plate, a spiral, or a torque spring to provide sufficient auxiliary elastic force and prevent excessive deformation of the elastic support bar and the situation that cannot be restored. However, elastic metal incurs a higher cost, and the manufacture of the elastic metal strip in form of a spiral or a torque spring has a larger volume, so that the thickness of the protector cannot be reduced, and it is uncomfortable to wear a thick protector.

In the aforementioned prior arts, the elastic metal strip is fixed to the joint protector and cannot be detached. After a long time of use, the joint protector will become dirty due to sweat or other factors and need cleaning. Obviously, it is relatively more difficult to clean the conventional joint protector, and the elastic metal strip may be damaged, oxidized or rusted.

In view of the drawbacks of the prior art, the inventor of the present invention based on years of experience in the related industry to conduct extensive researches and experiments, and finally designed and developed an auxiliary elastic device to overcome the drawbacks of the conventional protector with a high cost of the elastic support bar, a large volume of the protector, and uncomfortable to wear.

2. Summary of the Invention

Therefore, it is a primary objective of the present invention to provide an auxiliary elastic device for a joint protector, and the device is installed to the joint protector and configured to be corresponsive to an external side of the human joint, and the device can produce an auxiliary elasticity for the joint during human activities.

To achieve the aforementioned and other objectives, the present invention discloses an auxiliary elastic device comprising: a connecting body, coupled to a plurality of serially and pivotally coupled pivots; and a through slot, penetrating through the connecting body; and at least one stripe elastic rib, movably installed in the through slot; wherein both ends of the connecting body are inserted and sheathed on two positioning plates respectively, and the positioning plates are capable of mounting the connecting body to the joint protector, and the connecting body and the elastic rib are capable of bending with the human joint moving direction, and one of the elastic ribs is capable of providing an auxiliary elasticity of an elastic supporting force and an elastic restoring force when the human joint bends.

After a user wears the joint protector, the auxiliary elastic device is installed to an external side of the human joint, and the positioning plates at both ends of the auxiliary elastic device are installed to the external side of the user's thigh and the external side of the user's calf. When the user performs different exercises such as jogging, walking, squatting or getting up, the connecting body and elastic rib can be bent or straightened with the joint moving direction. Particularly, the elastic rib can provide an elastic supporting force and an elastic restoring force when the human joint bends, while achieving the effect of reducing the weight of the joint protector and the burden of the joint.

The present invention is elaborated by the following embodiment.

In an embodiment, the plurality of pivots of the connecting body further includes a central connector disposed at the mid-section position of the connecting body, and an external end of the pivot disposed separately at both ends of the connecting body is pivotally coupled to the fitting member that is sheathed on the two positioning plates separately, and the through slot penetrates through each pivot, the central connector and the two fitting members.

In an embodiment, the two positioning plates further include an inserted slot each formed in a direction facing both ends of the connecting body and provided for inserting and sheathing the fitting members, so that the connecting body can be mounted onto the joint protector conveniently through the insert or removed from the joint protector to facilitating cleaning the joint protector.

In an embodiment, two auxiliary positioning plates are extended outwardly from both sides of the central connector, and the two auxiliary positioning plates are fixable to the joint protector.

In an embodiment, the auxiliary elastic device further comprises a pivot tab extended from the pivoting position among the adjacent pivots, the central connector and the two fitting members, and every two adjacent pivot tabs are coupled to each other, and a shaft is passed and installed to the middle position of the two coupled pivot tabs, so that the two coupled pivot tabs can be deflected with an angle by using each shaft as the center, and the connecting body comprised of the plurality of pivots, the central connector, and the two fitting members is in a bending state.

In an embodiment, each pivot tab is in form of a circular plate, and a stop portion is protruded to the outside of the pivot tab, and after every two coupled pivot tabs are deflected for an angle, the stop portions of the two pivot tabs abut against each other to limit the angle of deflection between the stop portions, so as to limit the bending curvature of the connecting body and prevent a too-large bending curvature of the connecting body that may crack or break the elastic rib.

In an embodiment, the two positioning plates and the two auxiliary positioning plates are flexible fabrics or plastic cushions sewed onto the joint protector.

In an embodiment, the two positioning plates and the two auxiliary positioning plates are Velcro tapes for attaching the joint protector, so that the connecting body can be positioned quickly and securely to different models of joint protectors through the Velcro tapes.

In an embodiment, the elastic rib is made of a carbon fiber material or a glass fiber material, and such materials not just provide the elastic supporting force and the elastic restoring force when the user's joint bends only, but also facilitate the mass production and have the advantages of adjustable length, low production cost, small volume, and high water and corrosion resistance.

Compared with the prior art, the connecting body and the elastic rib of the present invention can be bent or straightened with the joint moving direction. Particularly, the elastic rib is made of a carbon fiber material or a glass fiber material and capable of providing an elastic supporting force and an elastic restoring force when the human joint bends, as well as achieving the effect of reducing the weight of the joint protector and the burden of the joint.

In addition, the two positioning plates may be flexible fabrics, plastic cushions, Velcro tapes or in form of insert slots that provide different fixation methods, and thus not just having the effect of facilitating the cleaning of the joint protector only, but also extending the scope of applicability of the auxiliary elastic device.

### BRIEF DESCRIPTION OF THE DRAWINGS

FIG. 1 is a schematic view of an installation position of the present invention;

FIG. 2 is a perspective view of the present invention;

FIG. 3 is an exploded view of the present invention;

FIG. 4 is a schematic view of an auxiliary elastic device of the present invention in a non-bending state;

FIG. 5 is a perspective view of FIG. 4;

FIG. 6 is a first schematic view of an auxiliary elastic device of the present invention in a bending state;

FIG. 7 is a second schematic view of an auxiliary elastic device of the present invention in a bending state;

FIG. 8 is a third schematic view of an auxiliary elastic device of the present invention in a bending state; and

FIG. 9 is a perspective view of FIG. 8.

### DESCRIPTION OF THE PREFERRED EMBODIMENTS

The above and other objects, features and advantages of this disclosure will become apparent from the following detailed description taken with the accompanying drawings.

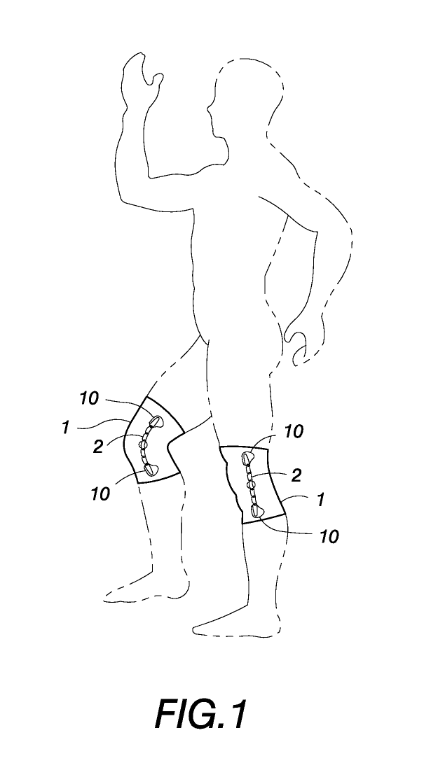

With reference to FIG. 1 for an auxiliary elastic device for a human joint in accordance with the present invention, the auxiliary elastic device is installed to a joint protector 1 and configured to be corresponsive to an external side of the human joint. The auxiliary elastic device comprises a connecting body 2, and two positioning plates 10 sheathed on both ends of the connecting body 2 respectively and provided for mounting the connecting body 2 onto the joint protector 1.

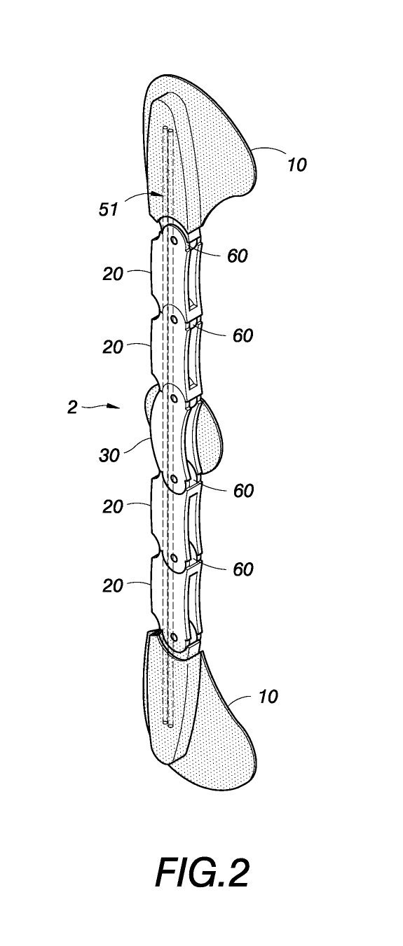

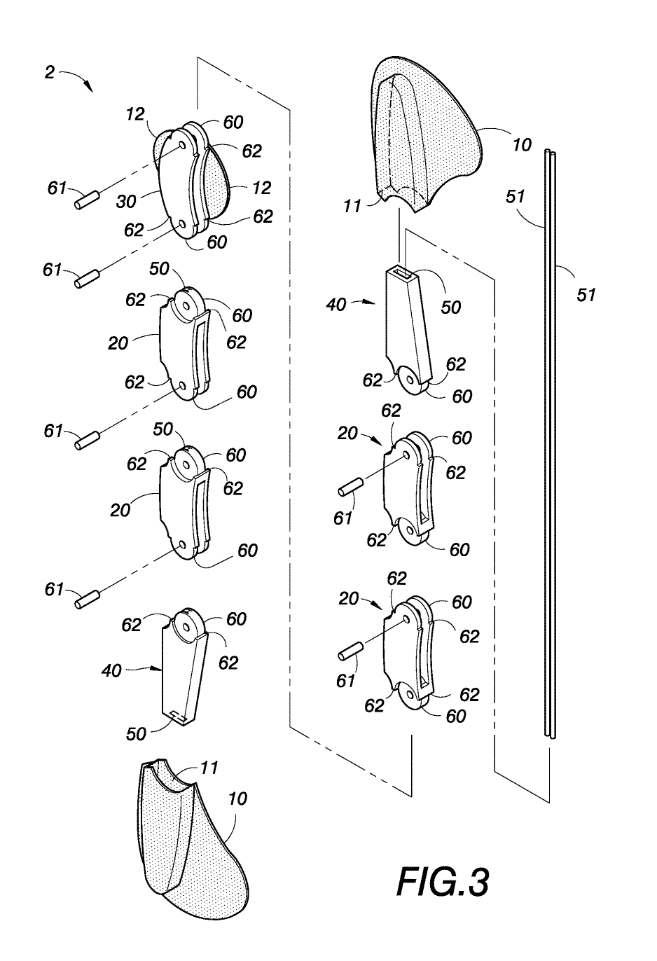

In FIGS. 1 to 3, the connecting body 2 comprises a plurality of serially and pivotally coupled pivots 20, a central connector 30 pivotally installed at a mid-section position of the plurality of pivots 20, and a fitting member 40 pivotally and separately coupled to the external side of both ends of the pivot 20 of the connecting body 2. The quantity of pivots 20 may be adjusted according to user requirements. The more the pivots 20, the longer the assembled connecting body 2.

The connecting body 2 includes a through slot 50 passing through each of the pivots 20, a central connector 30 and two fitting members 40, and at least one stripe elastic rib 51 is movably installed in the through slot 50, and the connecting body 2 and the elastic rib 51 can bend with the human joint moving direction. There may be a plurality of elastic ribs 51 when needed, and the elastic ribs 51 can provide an auxiliary elasticity of an elastic supporting force and an elastic restoring force when the human joint bends. In an embodiment, the elastic rib 51 may be made of a carbon fiber material or a glass fiber material to facilitate mass production, and its length may be adjusted according to the quantity of pivots 20. The elastic rib 51 has the advantages of low production cost, small volume, and high water and corrosion resistance.

The two positioning plates 10 further include an insert slot 11 formed in a direction facing both ends of the connecting body 2 and provided for inserting or sheathing the fitting member 40, so that the connecting body 2 can be installed to the joint protector 1 conveniently through the insert slot 11 or removed from the joint protector 1 conveniently to facilitate the cleaning of the joint protector 1.

In addition, the central connector 30 further has two auxiliary positioning plates 12 extended outwardly from both sides of the central connector 30, and the auxiliary positioning plates 12 may be fixed to the joint protector 1. In an embodiment, the two positioning plates 10 and the two auxiliary positioning plates 12 are flexible fabrics or plastic cushions sewed onto the joint protector 1 or Velcro tapes attached onto the joint protector 1, so that the connecting body 2 can be positioned quickly and securely onto different models of joint projectors 1 through the Velcro tape. In the figures, the two positioning plates 10 are plastic cushions, and each positioning plate 10 and its corresponsive insert slot 11 are integrally formed.

In an embodiment of the connecting body 2, a pivot tab 60 is extended from a pivoting position between the adjacent pivots 20, central connector 30, and two fitting members 40, and every two adjacent pivot tabs 60 are coupled to each other, and a shaft 61 is passed and installed at the middle position of two coupled pivot tabs 60, so that the two coupled pivot tabs 60 can be deflected with an angle by using each shaft 61 as the center, such that the connecting body 2 comprised of the plurality of pivots 20, the central connector 30 and the two fitting members 40 is in a bending state.

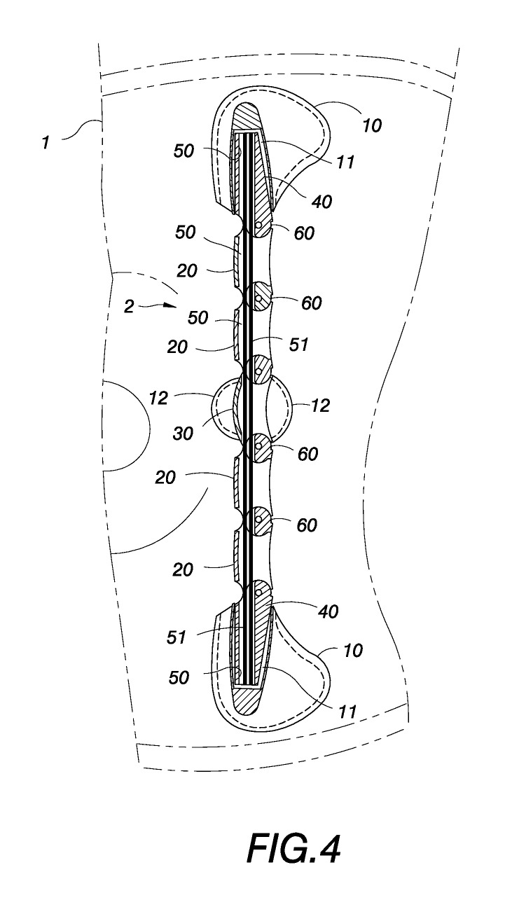

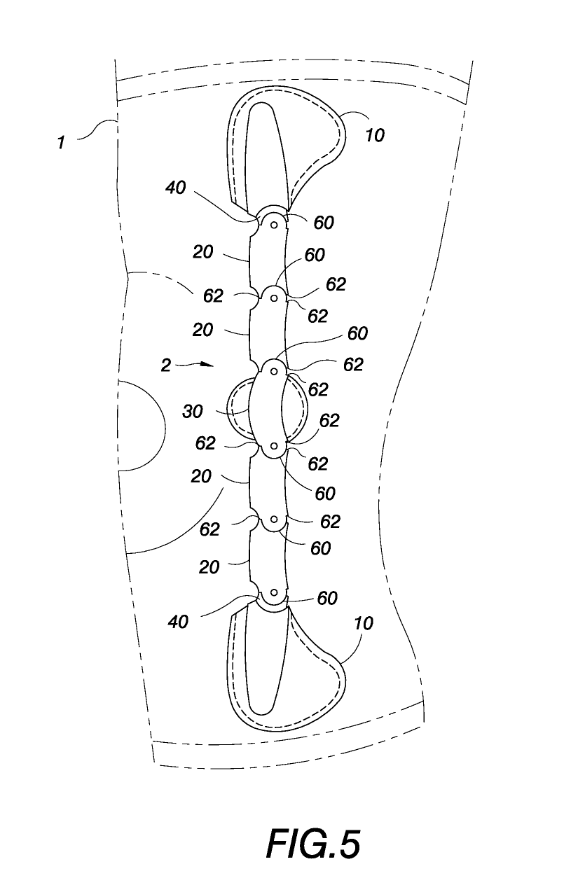

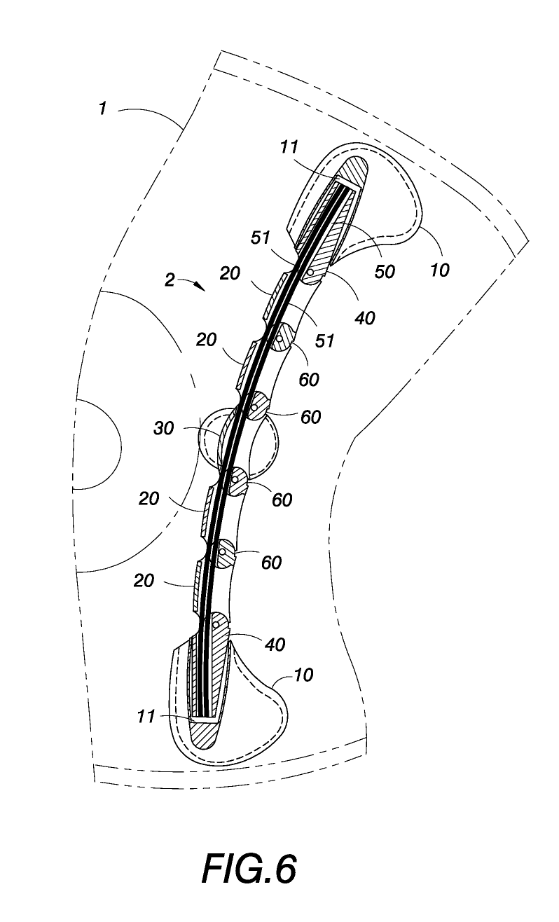

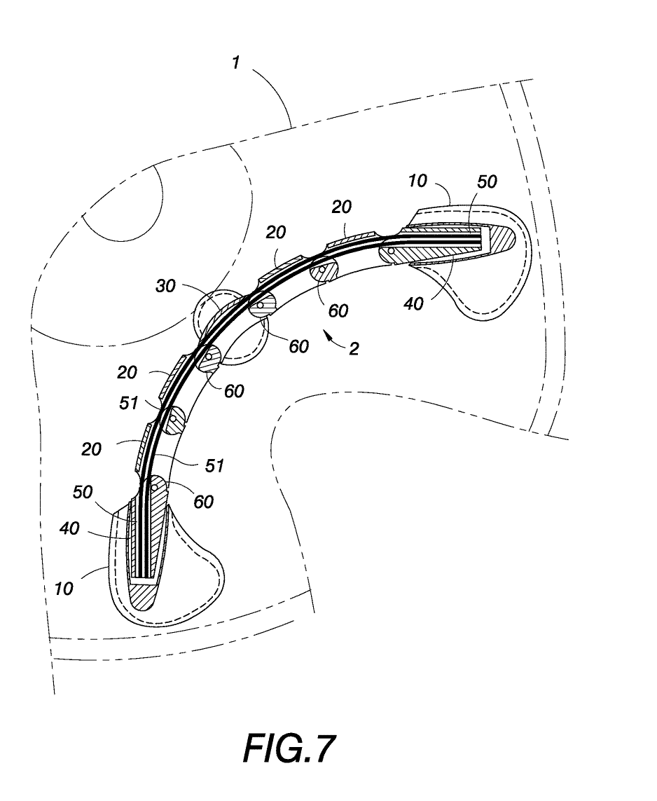

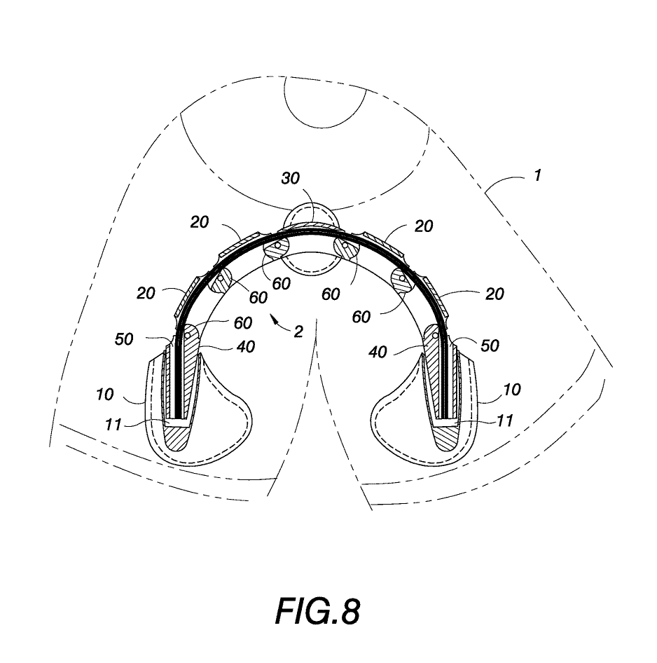

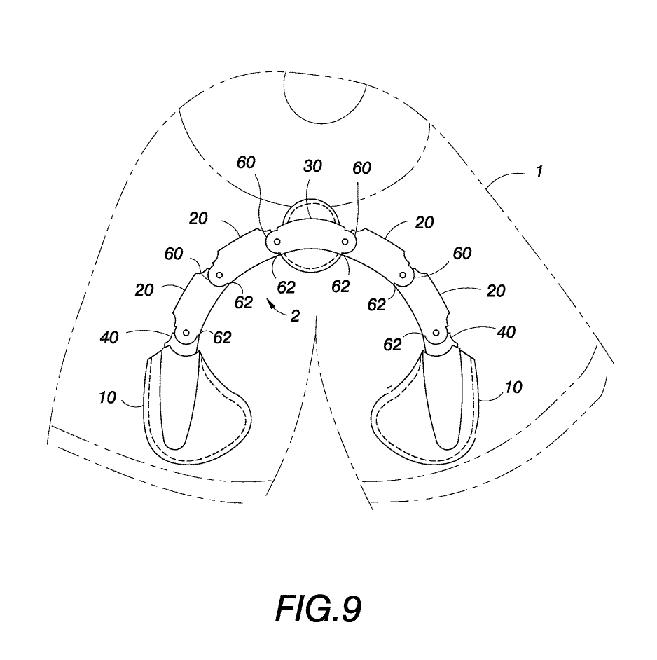

Each pivot tab 60 is in form of a circular plate and has a stop portion 62 protruded to the outside. With reference to FIGS. 4 and 5 for an auxiliary elastic device of the present invention in a non-bending state, the stop portions 62 of the two pivot tabs 60 abut against each other to restrict the connecting body 2 from bending in an opposite direction. In FIGS. 6 to 9, after the two coupled pivot tabs 60 are deflected with an angle, the stop portions 62 of the two pivot tabs 60 abut against each other to restrict the angle of deflection between the two, so as to limit the bending curvature of the connecting body 2 and prevent a too-large bending curvature of the connecting body 2 that may crack or break the elastic rib 51.

In the aforementioned structure, after a user has worn the joint protector 1, the auxiliary elastic device is installed to the external side of the human joint, and the positioning plates 10 at both ends are installed to the external side of a human thigh and the external side of a human calf respectively. When the user perform different types of exercises such as jogging, walking, squatting, or getting up, the connecting body 2 and the elastic rib 51 can be bent or straightened with the joint moving direction, and when the elastic rib 51 can an elastic supporting force and an elastic restoring force when the human joint bends. The invention also has the effect of reducing the weight of the joint protector 1 and the burden of the joint.

In addition, when the connecting body 2 is bent or straightened, the length of the connecting body 2 is changed, so that the fitting member 40 can move slightly in the insert slot 11 of the positioning plate 10 when the human joint is moving, so as to prevent the two positioning plates 10 from affecting the curvature angle of the connecting body 2.

What is claimed is:

1. An auxiliary elastic device for a joint protector, installed on an external side of the joint protector and configured to be corresponsive to a human joint, and comprising:
a connecting body, coupled to a plurality of serially and pivotally coupled pivots; and
a through slot, penetrating through the connecting body;
at least one stripe elastic rib, movably installed in the through slot;
wherein both ends of the connecting body are inserted and sheathed on two positioning plates respectively, and the positioning plates are capable of mounting the connecting body to the joint protector, and the connecting body and the elastic rib are capable of bending with the human joint moving direction, and one of the elastic ribs is capable of providing an auxiliary elasticity of an elastic supporting force and an elastic restoring force when the human joint bends.

2. The auxiliary elastic device for a joint protector according to claim 1, wherein the plurality of pivots of the connecting body further includes a central connector disposed at the mid-section position of the connecting body, and an external end of the pivot disposed separately at both ends of the connecting body is pivotally coupled to the fitting member that is sheathed on the two positioning plates separately, and the through slot penetrates through each pivot, the central connector and the two fitting members.

3. The auxiliary elastic device for a joint protector according to claim 2, wherein the two positioning plates further include an inserted slot each formed in a direction facing both ends of the connecting body and provided for inserting and sheathing the fitting members.

4. The auxiliary elastic device for a joint protector according to claim 2, wherein two auxiliary positioning plates are extended outwardly from both sides of the central connector, and the two auxiliary positioning plates are fixable to the joint protector.

5. The auxiliary elastic device for a joint protector according to claim 2, further comprising a pivot tab extended from the pivoting position among the adjacent pivots, the central connector and the two fitting members, and every two adjacent pivot tabs being coupled to each other, and a shaft being passed and installed to the middle position of the two coupled pivot tabs, so that the two coupled pivot tabs can be deflected with an angle by using each shaft as the center, and the connecting body comprised of the plurality of pivots, the central connector, and the two fitting members is in a bending state.

6. The auxiliary elastic device for a joint protector according to claim 5, wherein each pivot tab is in form of a circular plate, and a stop portion is protruded to the outside of the pivot tab, and after every two coupled pivot tabs are deflected for an angle, the stop portions of the two pivot tabs abut against each other to limit the angle of deflection between the stop portions, so as to limit the bending curvature of the connecting body.

7. The auxiliary elastic device for a joint protector according to claim 4, wherein the two positioning plates and the two auxiliary positioning plates are flexible fabrics or plastic cushions sewed onto the joint protector.

8. The auxiliary elastic device for a joint protector according to claim 4, wherein the two positioning plates and the two auxiliary positioning plates are Velcro tapes for attaching the joint protector.

9. The auxiliary elastic device for a joint protector according to claim 1, wherein the elastic rib is made of a carbon fiber material or a glass fiber material.
